(19) United States
(12) Patent Application Publication
(10) Pub. No.: US 2026/0284001 A1
(43) Pub. Date: Sep. 24, 2026

(54) NANOPARTICLES OF MYCOPHENOLIC ACID PRODRUG MOLECULES AND THERAPEUTIC USE THEREOF

(71) Applicant: A.D.A. S.R.L., Torino (IT)

(72) Inventors: Davide RUSSO, Torino (IT); Alessandro MARENGO, Torino (IT)

(21) Appl. No.: 19/474,506

(22) PCT Filed: Apr. 10, 2024
(86) PCT No.: PCT/IB2024/053489

§ 371 Date: Oct. 10, 2025

(30) Foreign Application Priority Data
Apr. 11, 2023 (IT) 102023000006933

(51) Int. Cl. A61K 31/343; A61K 9/107; A61K 47/60; A61P 37/06; B82Y 5/00; C07D 307/88

(52) U.S. Cl. CPC A61K 31/343; A61K 9/1075; A61K 47/60; A61P 37/06; C07D 307/88; B82Y 5/00

(57) ABSTRACT

A nanoparticle has a plurality of therapeutic molecules arranged in the form of a spherical micelle, suitable for localized delivery and release of mycophenolic acid, and effective in treatment of autoimmune diseases, fibrotic diseases and/or organ rejection diseases. A pharmaceutical composition including the nanoparticle in a pharmaceutically acceptable vehicle, and a method for manufacturing the nanoparticle are provided.

The present invention relates to the pharmaceutical and health field.

In particular, the invention concerns the delivery/formulation of active ingredients in nanoparticles with a micellar structure, more specifically mycophenolic acid, and their use for the treatment of autoimmune diseases, fibrotic diseases, and transplant rejection diseases, in particular affecting the lungs.

Mycophenolic acid is an immunosuppressive drug belonging to the 2-benzofuran class, used to prevent organ rejection in heart, kidney and liver transplants and to treat some autoimmune diseases. This compound is produced by Penicillium stoloniferum and operates by blocking de novo purine synthesis through the reversible and non-competitive inhibition of the enzyme Inosine Monophosphate Dehydrogenase (IMPDH). Inhibition of this enzyme leads to a reduction in lymphocyte proliferation, consequently making them less effective in recognizing and attacking the transplanted organ.

Mycophenolic acid (MPA) is predominantly administered as a sodium salt or mofetil ester, either orally in the form of capsules, tablets and suspensions, or intravenously. Usually used dosages are 180 mg, 360 mg and 500 mg for tablets, 250 mg for capsules, 1 g/5 ml as an oral suspension, and 500 mg for intravenous administration.

Following oral administration, the sodium salt and mofetil ester are absorbed mainly in the small intestine in the form of mycophenolic acid. In the case of mofetil ester there is a presystemic activation in the intestine by carboxylesterases (CES), which hydrolyse the ester into mycophenolic acid. A major first-pass metabolism is observed in the liver, which is mediated by glucoronosyltransferases which transform mycophenolic acid mainly into the inactive metabolite 7-O glucuronide.

As stated by Staatz et. al in “Clinical pharmacokinetics and pharmacodynamics of mycophenolate in solid organ transplant recipients”, Clin Pharmacokinet. 2007; 46(1): 13-58, this metabolite has plasma concentrations 20 to 100 times higher than mycophenolic acid (MPA). A percentage of this inactive metabolite, through biliary excretion, reaches the gastrointestinal tract again where it is converted back to mycophenolic acid, resulting in a second blood peak between 6 and 12 hours. To a lesser extent, three other metabolites are formed, including the acyl-glucoronide derivative, which has a pharmacological potency comparable to MPA. Excretion of MPA occurs mainly through urine (87%), in the form of the inactive metabolite 7-O glucoronide.

With regard to the dosage, mycophenolate mofetil is generally administered at doses of 2 g/day for kidney transplantation, 3 g/day for heart transplantation, and 3 g/day for liver transplantation. When used other than authorized (off label) for the treatment of lupus nephritis, the doses range from 0.5 to 3 g/day, while for interstitial lung disease the doses range from 1 to 3 g/day.

The high doses of systemic therapy that are necessary to compensate for the first-pass effect and achieve a therapeutic concentration of MPA in the target organ or tissue are associated with the appearance of major side effects mainly in the gastrointestinal and genitourinary tract. Furthermore, the resulting high total immunosuppressive load may lead to the onset of opportunistic infections, fatal infections, sepsis, and blood disorders (neutropenia or anemia).

The chemical structure of the mycophenolic acid and mofetil ester molecules exhibits a phenolic group suitable for use in an esterification reaction. The scientific paper by Xie H. et al. (“Target-oriented delivery of selfassembled immunosuppressant cocktails prolongs allogeneic orthotopic liver transplant survival”, Journal of Controlled Release, 2020, Volume 328:237-250) describes self-assembling structures obtained through the esterification of the phenolic group of mycophenolate mofetil with alcohols of different chain lengths. Noteworthy, these nanoparticles have a morpholine ring, the nitrogen atom of which, under acidic pH conditions, such as the pH in tissues and biological fluids in which an inflammatory process is under way, is protonated. pH-dependent protonation of nanoparticles having morpholine structures has also been reported by Zhang Y. et al., “Morpholino-decorated long circulating polymeric micelles with the function of surface charge transition triggered by pH changes”, Chem Commun (Camb); 2013, 49(66): 7286-8.

The therapeutic application of positively charged nanoparticles, however, has several limitations, especially in the field of lung diseases, mainly due to the absence of mucus-penetrating properties, as described in the Article by Lai K. et al., “Mucus-penetrating nanoparticles for drug and gene delivery to mucosal tissues”; 2009, Adv Drug Deliv Rev., 61(2): 158-71, as well as the dose-dependent irritant effect on the airways caused by changes in osmolality of lung fluids (Son Y. J. et al., in “Optimizing spray-dried porous particles for high dose delivery with a portable dry powder inhaler”; 2021, Pharmaceutics. 13(9):1528).

In addition, protonation of the nanoparticles can cause structural change thereto caused by the repulsion between the positive charges of the monomers that make them up, resulting in loss of the nanoparticle structure as described by Mu Y. et al. in “Advances in pH-responsive drug delivery systems”; 2021, OpenNano and Nakayama M. et al. in “Polymeric micelles with stimuli-triggering systems for advanced cancer drug targeting”; 2014, J Drug Target., 22(7): 584-99. However, this phenomenon, if not properly researched, can adversely affect the effectiveness of the therapy as it causes the release of the active ingredient in an uncontrolled manner before it reaches the target site, as reported by Alsawaftah N. et al. in “pH responsive nanocarriers in cancer therapy”; 2022, Polymers., 14(5): 936.

Mycophenolic acid, and the mofetil ester and sodium salt thereof show poor solubility in aqueous medium (0.0355 mg/ml for mycophenolic acid, 0.043 mg/ml for mycophenolate mofetil, 0.057 mg/ml for mycophenolic acid sodium salt-Drugbank source), which requires the use of solubilizing agents.

To overcome the limitation of the poor solubility of mycophenolic acid, several formulation strategies have been developed, which involved the use of nanoparticle systems, such as liposomes (Patel et. al, “Liposomal delivery of Mycophenolic Acid with Quercitin for improved breast cancer therapy in SD rats”, Front Bioeng Biotechnol. 2020 Jun. 16; 8:631), cyclodextrins (Khalafi et. al, “Investigation of the inclusion complex of beta-cyclodextrin with Mycophenolate mofetil”, Spectrochim Acta A Mol Biomol Spectrosc. 2012 May; 90:45-9; Iqbal et. al, “Solid lipid nanoparticles of Mycophenolate mofetil: an attempt to control the release of an immunosuppressant”, Int J Nanomedicine. 2020 Aug. 5; 15:5603-5612) and polymeric nanoparticles (Shirali et. al “Nanoparticles delivery of Mycophenolic acid upregulates PD-L1 on dendritic cells to prolong murine allograft survival”, Am J Transplant. 2011 December; 11(12): 2582-92), with the aim of improving its aqueous solubility.

The above strategies therefore involve the encapsulation of MPA within excipients capable of forming nanostructures. However, this approach has major limitations, mainly due to the need to use an excess of excipient in relation to the active ingredient, resulting in low drug loading levels. Furthermore, nanostructure systems as described above may be subject to phenomena of initial release of the active ingredient contained therein at higher rates than steady-state, the so-called “burst release” phenomenon, which may affect the effectiveness of the treatment.

In order to achieve a more efficient system for delivering mycophenolic acid to target tissues or organs in the context of its therapeutic use, for example for the treatment of autoimmune, fibrotic and organ rejection diseases, in particular affecting the lungs, which is capable of overcoming the limitations of the formulation strategies of the prior art as previously illustrated, the present inventors manufactured a new nanoparticle comprising a plurality of therapeutic molecules containing mycophenolic acid.

Therefore, a first aspect of the invention is a nanoparticle comprising a plurality of therapeutic molecules arranged in the form of a spherical micelle, said therapeutic molecules having the general formula (I)

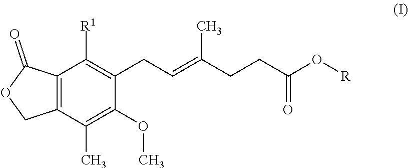

wherein R is a linear or branched, saturated or unsaturated, alkyl chain containing from 5 to 22 carbon atoms, and
R¹ is-OH, optionally conjugated with a conjugation molecule selected from the group consisting of polyethylene glycol (PEG), triethylene glycol (TEG), hyaluronic acid, sugars such as mannose and trehalose, proteins, peptides, antibodies, messenger RNAs (mRNAs), small interfering RNAs (siRNAs), microRNAs (miRNAs), aptamers, and any combination thereof.

A pharmaceutical composition comprising a nanoparticle as defined above and the therapeutic use of said nanoparticle or pharmaceutical composition, as defined in the attached independent claims, are further objects of the invention.

Additional features and advantages of the invention are defined in the dependent claims, which form an integral part of the specification.

In the context of the present description, the term “in the form of a spherical micelle” is intended to mean a spherical structure resulting from the clustering of the plurality of the therapeutic molecules as defined above so that the hydrophobic alkyl chains of said molecules lie within the spherical structure, thus forming the so-called micellar “core”, and the hydrophilic groups corresponding to the hydroxyphthalide portion of the therapeutic molecules are oriented towards the outside of said structure, thus forming the so-called micellar “corona”.

Preferably, in the therapeutic molecules of the nanoparticle according to the invention R is a linear or branched, saturated or unsaturated alkyl chain containing from 5 to 20 carbon atoms, preferably from 8 to 17 carbon atoms, e.g., 8, 9, 10, 11, 12, 13, 14, 15, 16 or 17 carbon atoms.

Most preferably, R is a saturated linear alkyl chain containing 16 carbon atoms.

According to the invention, the R¹ group in the therapeutic molecule of the nanoparticle is optionally conjugated with a conjugation molecule as defined above.

In the context of the present invention, the term “conjugated” refers to the presence of a covalent ester bond between the R¹ group of the therapeutic molecule and a carboxyl group, or a derivative thereof, of the conjugation molecule, whether or not preceded by a spacer.

Therefore, in one embodiment of the invention, the R¹ group is directly conjugated to the conjugation molecule, e.g., hyaluronic acid, by forming an ester bond between said R¹ group and the one or more carboxyl groups of the hyaluronic acid.

The presence of hyaluronic acid on the surface of the nanoparticle of the invention advantageously allows the targeting of the nanoparticle to cells expressing the intercellular adhesion molecule CD44.

Optionally, the conjugation molecule, for example polyethylene glycol or a sugar such as mannose, can be functionalized with one or more functional groups suitable to form an ester bond, e.g., with one or more carboxyl groups or with one or more acyl groups. Non-limiting examples of functionalized conjugation molecules suitable for use in the present invention are heterobifunctional polyethylene glycol (OH-PEG-COOH), m-PEG-acyl chloride, 5-(alpha-di-mannopyranosyl-oxy)pentanoic acid (Alpha-Man-Bu-COOH) and (((alpha-di-mannopyranosyloxy)ethoxy)ethoxy)propionic acid (Alpha-Man-TEG-COOH).

In another embodiment, the R¹ group is conjugated to the conjugation molecule via a bifunctional spacer. According to this embodiment, the bifunctional spacer comprises a first functional group suitable to form a covalent bond with the conjugation molecule, and a second functional group, for example a carboxyl group or a derivative thereof, suitable to form an ester bond with the R¹ group.

Bifunctional spacers suitable for use in the conjugation of the R¹ group with a protein conjugation molecule include, but are not limited to, maleimide-PEG-acetic acid (Mal-PEG-COOH), which is capable of reacting with the thiol group present on the protein molecule.

The conjugation between the R¹ group and a conjugation molecule selected from messenger RNA (mRNA), small interfering RNA (siRNA), microRNA (miRNA) and aptamer may be achieved by using a bifunctional spacer comprising (i) a functional group capable of reacting, for example, with the amino group of an aptamer (modified aptamers with an amino group at the 5′ end) and (ii) a carboxyl group capable of reacting with the R¹ group. An example of a bifunctional spacer having the above characteristics is N-hydroxysuccinimide-PEG-COOH. Another spacer suitable for the conjugation described above is 2-aminobutyl-1,3-propanediol.

As will be apparent from the following detailed description, the present invention provides a new nanoparticle with a micellar structure composed of a plurality of prodrug molecules obtained by esterification of mycophenolic acid with long-chain alcohols, capable of delivering said mycophenolic acid and releasing it in a localized manner.

A “prodrug”, as used herein, refers to any medicament that is inactive until properly activated/metabolized within the body.

The present invention is based, in fact, on the surprising finding by the inventors that mycophenolic acid prodrug molecules as defined above, despite their high lipophilicity and the absence of an amphiphilic structure, can cluster with each other spontaneously in water to form micellar structures (self-assembly), without the aid of additional substances.

The present inventors also noted a surprising increase in the apparent water solubility of the nanoparticles of the present invention compared to the water solubility limit of mycophenolic acid (at least eight times).

This property of the nanoparticles of the invention is completely unexpected in the light of the teachings of the prior art. By way of example, the Article by Sifei Han, (Han S. et al, “Targeted delivery of a model immunomodulator to the lymphatic system: comparison of alkyl ester versus triglyceride mimetic lipid prodrug strategies”; 2014 J Control Release 177:1-10) describes the use of MPA lipophilic esters to increase the passive distribution thereof in lipids and lipoproteins in the lymphatic circulation.

Patent application WO 2017/019636 describes lipophilic prodrug molecules of mycophenolic acid and nanoparticle systems for the delivery thereof, such as for example liposomes and micelles, in which the prodrug molecules are however complexed with or encapsulated within the one or more lipid components of the nanoparticles.

In vitro studies carried out by the same inventors have also shown that nanoparticles having the characteristics as defined above advantageously have a remarkable ability to penetrate through thick mucous secretions such as the bronchial mucus.

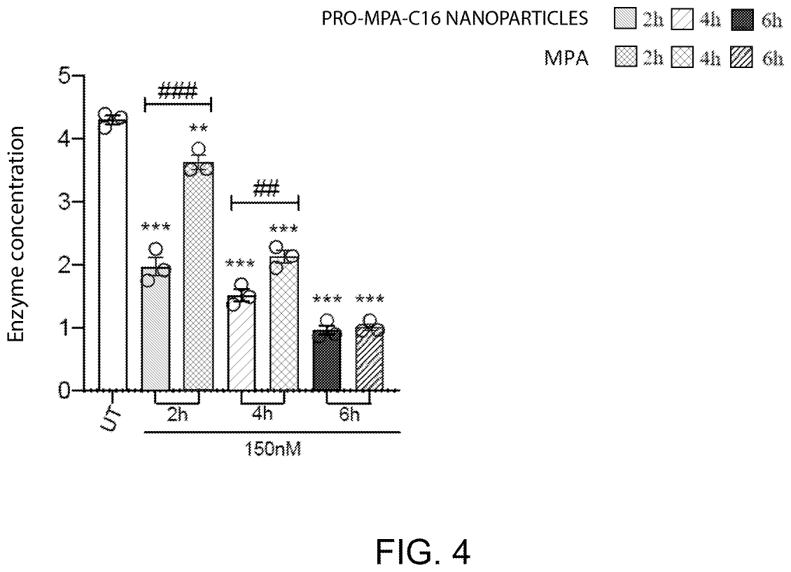

As shown in FIG. 4, following treatment of human bronchial epithelial cells with the nanoparticles of the invention in the presence of a layer of sputum from patients suffering from bronchiectasis/cystic fibrosis, which is known to be particularly thick and viscous, these cells exhibited a reduction in the levels of the target enzyme Inosine Monophosphate Dehydrogenase (IMPDH) significantly higher than the reduction achieved with mycophenolic acid as such, used as a control.

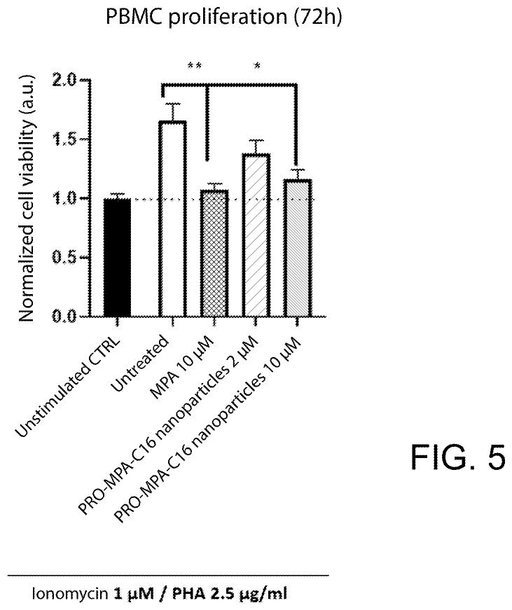

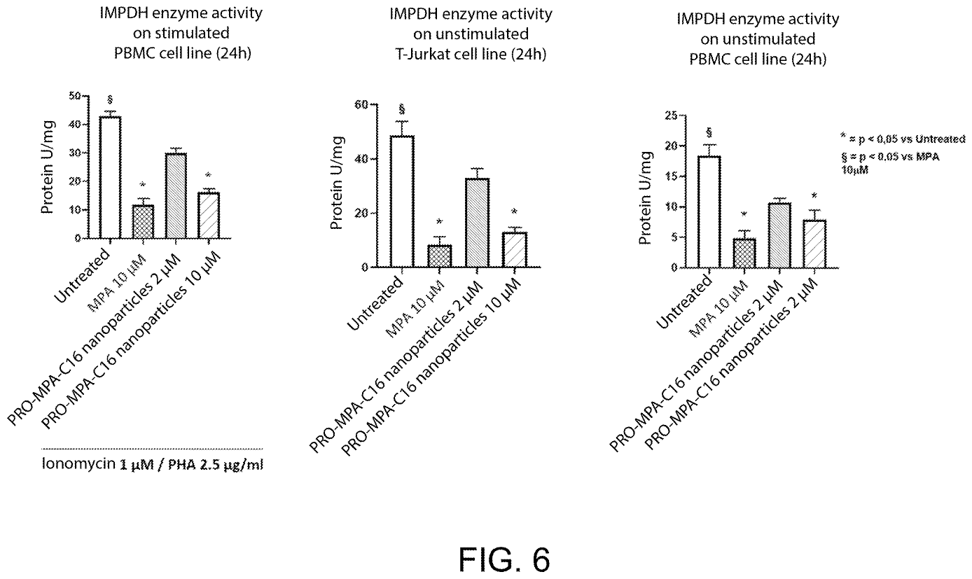

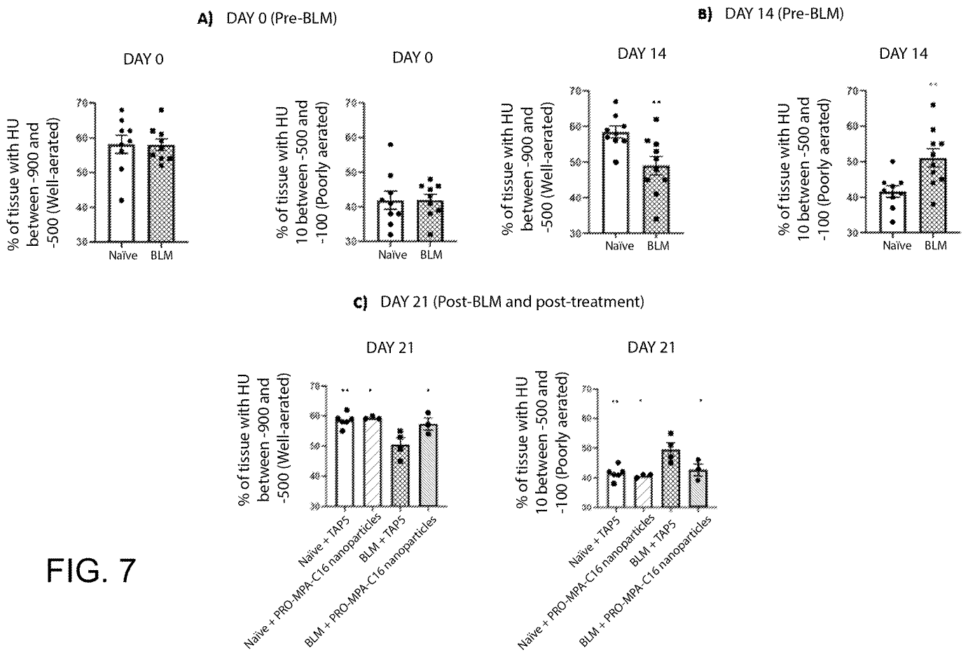

These surprising findings not only make it clear that the nanoparticles of the invention are able to permeate mucous secretions of complex density and viscosity in a particularly effective way, even without the need for additional aid from excipient ingredients, such as, for example, polyethylene glycol, but clearly indicate that said nanoparticles preserve at the same time their integrity and their ability to release the active ingredient mycophenolic acid in a localized manner. Further studies have also confirmed that, by reducing the levels of the IMPDH enzyme, the nanoparticles of the invention exert an inhibitory activity on the proliferation of immune cells, and that this inhibitory activity is dose-dependent (FIGS. 5 and 6). According to the results of the in vitro tests, the therapeutic activity of the nanoparticles of the invention was subsequently also validated in vivo, by using a mouse model of interstitial lung disease (SSc-ILD model), demonstrating that treatment of SSc-ILD animals by inhalation with the nanoparticles of the invention results in a significant improvement in the aeration state of the lung tissues (FIG. 7A-C).

Without wishing to be bound by any theory, the present inventors believe that the ability demonstrated by the nanoparticles according to the invention to cross physiological barriers, such as the dense secretions of the airways, could be attributable to the particular surface structure of the nanoparticles which expose the hydroxyphthalide component to the outside.

In the light of the above, the nanoparticles according to the invention therefore represent an important innovative therapeutic tool, particularly in the field of lung diseases.

According to one embodiment of the invention, the nanoparticle has a diameter ranging from 30 to 700 nm, preferably from 60 to 200 nm, still more preferably from 70 to 150 nm.

According to another embodiment, the zeta potential of the nanoparticle object of the invention ranges from −5 mV to −60 mV.

The term “zeta potential”, as used herein, refers to the charge that develops at the interface between a solid surface and the liquid in which it is immersed.

An important advantage of the nanoparticles of the present invention is that they do not require the use of additives such as emulsifiers and surfactants to ensure and/or facilitate the clustering of the mycophenolic acid prodrug molecules to form spherical micellar structures and to maintain the stability of said nanoparticles in an aqueous suspension.

It follows that the nanoparticle according to the present invention appears to contain and deliver a very high amount of mycophenolic acid, i.e., up to approximately 80% by weight of the total weight of the nanoparticle.

In addition, the absence of the above-mentioned additives implies that said nanoparticles are more suitable for all applications, in particular therapeutic applications, which require that the nanoparticles are non-toxic and stable in physiological media.

Therefore, according to one embodiment, the nanoparticle of the invention consists of a plurality of therapeutic molecules as defined above arranged in the form of a spherical micelle.

Although the nanoparticles according to the invention do not require the use of additives, the scope of the present invention also includes an embodiment in which said nanoparticles may comprise, in addition to the plurality of therapeutic molecules, a surfactant, preferably a surfactant with a low or no toxicity index. According to this embodiment, the presence of the surfactant allows certain chemical-physical properties of the nanoparticle of the invention to be improved, for example the size and the stability in biological fluids to be reduced and improved, respectively.

Exemplary surfactants suitable for use in the particle object of the present invention include, but are not limited to, phospholipids, polyethylene glycol (PEG) conjugates with lipophilic molecules such as the PEG-cholesterol conjugate, polyoxyethylene and polyoxypropylene copolymers, fatty acids esterified with ethoxylated sorbitan (polysorbates), and any combination thereof.

According to the present invention, the nanoparticle comprising or consisting of a plurality of therapeutic molecules as defined above may also internally contain, in the hydrophobic core consisting of the alkyl chains of said therapeutic molecules, a lipophilic therapeutic agent.

Advantageously, in this embodiment, the nanoparticle of the present invention is able to deliver, in addition to mycophenolic acid, further active ingredients for the treatment of a specific disease.

Lipophilic therapeutic agents suitable to be included in the nanoparticle of the invention include, but are not limited to, immunosuppressive agents such as, for example, tacrolimus, cyclophosphamide, everolimus, sirolimus, methotrexate, azathioprine, anti-inflammatory agents such as, for example, dexamethasone, budesonide, beclomethasone, antifibrotic agents such as pirfenidone and nintedanib, antitumor agents such as, for example, saracatinib.

The lipophilic therapeutic agent may be incorporated into the nanoparticles according to the invention by non-covalent encapsulation methods such as, for example, nanoprecipitation.

The selection of the most suitable encapsulation method for use within the scope of the present invention falls well within the skills of those of ordinary skill in the art.

Due to the beneficial chemical-physical properties described above and the surprising ability to penetrate through dense secretions such as the bronchial mucus, the nanoparticles according to the present invention are particularly suitable for therapeutic use, in particular for the treatment of autoimmune, fibrotic and organ rejection diseases, in particular affecting the lungs.

In a preferred embodiment, the nanoparticles according to the present invention are suitable for therapeutic use for the treatment of an autoimmune lung disease and/or a fibrotic lung disease.

In another preferred embodiment, the nanoparticles according to the present invention are suitable for use for the therapeutic treatment and/or prevention of organ rejection, preferably of lung transplant rejection.

Autoimmune lung diseases and/or fibrotic lung diseases that can be treated with the nanoparticles according to the invention include, for example, but are not limited to, systemic sclerosis-associated interstitial lung disease (SSc-ILD), lymphangioleiomyomatosis (LAM), sarcoidosis and bronchiolitis obliterans syndrome (BOS).

A pharmaceutical composition comprising a nanoparticle of the invention as defined above, in combination with at least one pharmaceutically acceptable vehicle, excipient and/or diluent, is also within the scope of the invention.

According to the invention, the pharmaceutical composition is suitable for use in the above therapeutic medical applications relating to the nanoparticle according to the invention.

The pharmaceutical composition of the present invention can be formulated into any suitable dosage form, for example for administration via the enteral (oral or gastro-enteral, rectal, sublingual), parenteral (intravenous, intraarterial, transcutaneous, intramuscular, intradermal, subcutaneous, intraperitoneal), topical (direct contact of the drug with the site of action and/or the skin and/or the mucous membranes), ocular, inhalation, and intratracheal routes.

Preferred oral dosage forms are tablets, capsules, sachets, powders, granules, pellets, gels, syrups, elixirs, oral solutions, suspensions or emulsions. These dosage forms normally also include additional substances such as, for example, lactose, dextrose, mannitol, stearic acid, starch, and/or gelatin.

Injectable compositions, for example injectable solutions or suspensions in aqueous or oily solution, can be formulated according to the prior art and optionally using appropriate dispersing, wetting and/or suspending agents.

Suitable formulations for intranasal administration are, for example, powders with a particle size ranging from 10 to 150 μm, which include the nanoparticles of the invention as the active ingredient. These formulations may be administered, for example, by rapid inhalation through the nasal passages from a container of said powder held close to the nostrils.

Of course, the selection of suitable vehicles, excipients and/or diluents is carried out depending on the desired form of administration and this selection is within the skills of those of ordinary skill in the art. The selection of the dose of active ingredient and the dosage regimen also falls within the skills of those of ordinary skill in the art, and the selection thereof depends on several factors, such as for example the age and weight of the patient, as well as the degree of progression of the disease.

According to one embodiment of the invention, the pharmaceutical composition comprises at least one additional active ingredient. Non-limiting examples of the at least one additional active ingredient include immunosuppressive molecules such as, for example, everolimus, tacrolimus, methotrexate, sirolimus, cyclosporine, cyclophosphamide, azathioprine, anti-inflammatory molecules such as, for example, dexamethasone, budesonide, beclomethasone, antifibrotic agents such as, for example, pirfenidone and nintedanib, and antitumor agents such as, for example, saracatinib.

A further object of the present invention is a method for the production of a nanoparticle according to the invention, comprising the steps of:
(i) mixing a plurality of therapeutic molecules as defined above in a polar solvent, thereby obtaining a first mixture, wherein said polar solvent is not water;
(ii) adding the first mixture of step (i) to a predetermined amount of water under stirring, so as to obtain a second mixture, wherein the predetermined amount of water is at least 65% by volume of the total volume of the second mixture; and
(iii) removing the polar solvent from the second mixture of step (ii), thereby obtaining a nanoparticle comprising a plurality of therapeutic molecules arranged in the form of a spherical micelle, in an aqueous suspension.

The most preferred polar solvent is ethanol.

In the method according to the invention, the polar solvent can be removed by using the vacuum evaporation or dialysis method.

The selection of the most appropriate method for use within the scope of the present invention for evaporating the polar solvent falls well within the skills of those of ordinary skill in the art.

In a preferred embodiment of the invention, the critical micelle concentration (CMC) of the therapeutic molecules in the aqueous suspension of step (iii) has a value ranging from 50 to 150 ng/ml, preferably from 50 to 100 ng/ml.

The term “critical micelle concentration”, as used herein, refers to the concentration value of a solution of molecules at which a number of said molecules cluster into micelles.

The method of the invention may also comprise the step of separating the therapeutic molecules that did not participate in the formation of the nanoparticles from the aqueous suspension of step (iii).

Methods suitable for use in the above separation step include, but are not limited to, sedimentation, filtration and centrifugation procedures.

The centrifugation procedure is particularly preferred.

The preferred embodiments described above can be combined with each other as required, and the implementation of these combinations falls within the skills of the person skilled in the art.

The examples that follow are provided for illustration purposes and do not limit the scope of the invention as defined in the appended claims.

### EXAMPLES

### Example 1: Chemical-Physical Characterization of the Nanoparticle of the Invention

The present inventors conducted dedicated tests with the aim of characterizing the nanoparticles object of the invention according to various physical-chemical properties as described below.

### 1. Apparent Solubility

Apparent solubility refers to the solubility of a solute in a solvent, as measured under certain experimental conditions. In this specific case, the apparent solubility represents the range of concentrations at which stable aqueous suspensions of the nanoparticles of the invention can be formed. Stable suspensions refer to aqueous suspensions, composed of nanoparticles object of the invention and having a concentration higher than the aqueous solubility limit of mycophenolic acid and its two derivatives mofetil and sodium salt, where no phase separation and/or precipitate is observed at the end of the method of preparation thereof, and where stability, as described, is achieved without the aid from at least one stabilizing and/or solubilising excipient.

### 2. Size

The analysis was performed using the Zeta-sizer Nano Z instrument (Malvern Instruments, Malvern, UK). Measurements were taken at 25° C. in milliQ water. The technique used to measure the diameter of the nanoparticles is called Dynamic Light Scattering (DLS). This technique allows the hydrodynamic diameter of nanoparticles dispersed in a liquid to be measured. The sample is illuminated by a laser beam, and changes in intensity of the light scattered by the sample are measured as a function of time. The intensity changes measured by the detector are generated by the Brownian motion of the particles. At the same temperature and viscosity, small particles move rapidly creating rapid changes in the scattering intensity, whereas larger particles move more slowly creating slow changes in the intensity.

### 3. Zeta Potential

The analysis was performed using the Zeta-sizer Nano Z instrument (Malvern Instruments, Malvern, UK). All samples were analysed at 25° C. in milliQ water. The zeta potential of the nanoparticles of the invention was determined by measuring the electrophoretic mobility of the particles suspended in water. To measure the electrophoretic mobility of the particles, an electric field was applied between the electrodes of the measuring cell which holds the sample and is illuminated by a laser beam. The charged particles move towards the opposite-sign electrode, creating a change in frequency of the light scattered by the sample, which is directly proportional to the electrophoretic mobility.

### Example 2: Preparation of Mycophenolic Acid Prodrug Therapeutic Molecules

Four different therapeutic molecules of Formula (I) were prepared, which differ from each other in the length of the alkyl chain R, as follows:
(i) Pentyl (4E)-6-(1,3-dihydro-4-hydroxy-6-methoxy-7-methyl-3-oxo-5-isobenzofuranyl)-4-methyl-4-hexenoate, hereinafter referred to as PRO-MPA-C5;
(ii) Octyl (4E)-6-(1,3-dihydro-4-hydroxy-6-methoxy-7-methyl-3-oxo-5-isobenzofuranyl)-4-methyl-4-hexenoate, hereinafter referred to as PRO-MPA-C8;
(iii) Dodecyl (4E)-6-(1,3-dihydro-4-hydroxy-6-methoxy-7-methyl-3-oxo-5-isobenzofuranyl)-4-methyl-4-hexenoate, hereinafter referred to as PRO-MPA-C12;
(iv) Hexadecyl (4E)-6-(1,3-dihydro-4-hydroxy-6-methoxy-7-methyl-3-oxo-5-isobenzofuranyl)-4-methyl-4-hexenoate, hereinafter referred to as PRO-MPA-C16.

The different prodrug molecules were prepared through a synthetic process comprising two distinct steps:
formation of MPA acyl chloride;
reaction with alkyl alcohol to give the desired ester.

In the esterification reaction of the MPA carboxylic acid group with the alcoholic group of the alcohol, alkyl alcohols of different lengths were used:
pentanoic alcohol (C5);
octanoic alcohol (C8);
dodecanoic alcohol (C12);
hexadecanoic alcohol (C16).

### PRO-MPA-C5 Synthesis

For the synthesis of the molecule pentyl (4E)-6-(1,3-dihydro-4-hydroxy-6-methoxy-7-methyl-3-oxo-5-isobenzofuranyl)-4-methyl-4-hexenoate (PRO-MPA-C5), the following method was followed:

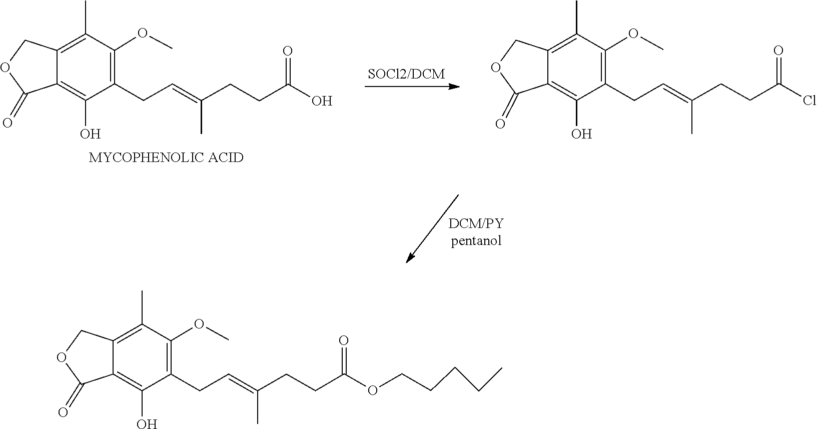 [SMILES: COc1c(C)c2c(c(O)c1C/C=C(\C)CCC(=O)O)C(=O)OC2 ; COc1c(C)c2c(c(O)c1C/C=C(\C)CCC(=O)Cl)C(=O)OC2 ; CCCCCOC(=O)CC/C(C)=C/Cc1c(O)c2c(c(C)c1OC)COC2=O]

In particular, mycophenolic acid (700 mg; 2.18 mmol) was dissolved in anhydrous dichloromethane (20 ml). Thionyl chloride (0.48 ml; 3 mol/eq) was added and the solution was stirred at room temperature for 5 hours. The solvent was removed under vacuum. The residue was dissolved in 10 ml of anhydrous dichloromethane and added dropwise to a solution of 1-pentanol (191 mg; 1 mol/eq) in anhydrous dichloromethane (5 ml) and pyridine (1.75 ml; 10 mol/eq.). The resulting mixture was stirred at room temperature for 12 hours under nitrogen atmosphere, then checked by HPLC-MS. Water was added, the two phases were separated, and the organic phase was further washed with 4% HCl solution, water and brine. The solvent was removed, and the residue was purified by chromatography (Isolera Biotage; Sfar column; eluent: hexane/ethyl acetate 9/1). Overall yield: 15%.

### PRO-MPA-C8 Synthesis

The molecule octyl (4E)-6-(1,3-dihydro-4-hydroxy-6-methoxy-7-methyl-3-oxo-5-isobenzofuranyl)-4-methyl-4-hexenoate (PRO-MPA-C8) was synthesized by the inventors using the following method:

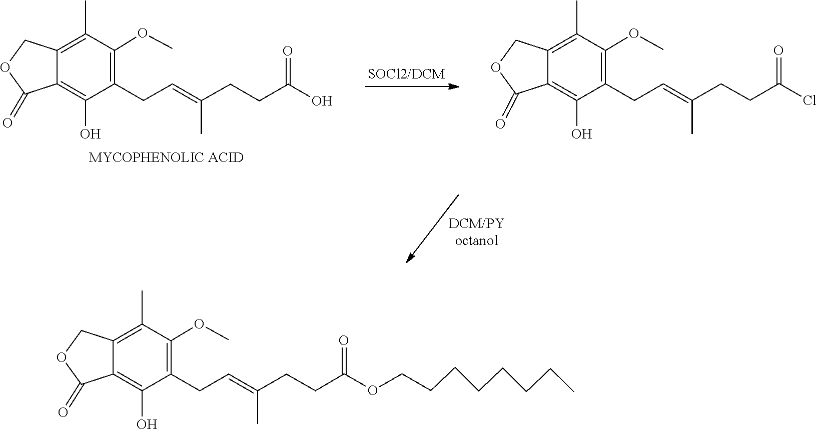 [SMILES: COc1c(C)c2c(c(O)c1C/C=C(\C)CCC(=O)O)C(=O)OC2 ; COc1c(C)c2c(c(O)c1C/C=C(\C)CCC(=O)Cl)C(=O)OC2 ; CCCCCCCCOC(=O)CC/C(C)=C/Cc1c(O)c2c(c(C)c1OC)COC2=O]

Mycophenolic acid (700 mg; 2.18 mmol) was dissolved in anhydrous dichloromethane (20 ml). Thionyl chloride (0.48 ml; 3 mol/eq) was added and the solution was stirred at room temperature for 5 hours. The solvent was removed under vacuum. The residue was dissolved in 10 ml of anhydrous dichloromethane and added dropwise to a solution of 1-octanol (283 mg; 1 mol/eq) in anhydrous dichloromethane (5 ml) and pyridine (1.75 ml; 10 mol/eq). The resulting mixture was stirred at room temperature for 12 hours under nitrogen atmosphere, then checked by HPLC-MS. Water was added, the two phases were separated, and the organic phase was further washed with 4% HCl solution, water and brine. The solvent was removed, and the residue was purified by chromatography (Isolera Biotage; Sfar column; eluent: hexane/ethyl acetate 9/1). Overall yield: 19%.

### PRO-MPA-C12 Synthesis

The synthesis of the molecule dodecyl (4E)-6-(1,3-dihydro-4-hydroxy-6-methoxy-7-methyl-3-oxo-5-isobenzofuranyl)-4-methyl-4-hexenoate (PRO-MPA-C12) was achieved with the method illustrated below:

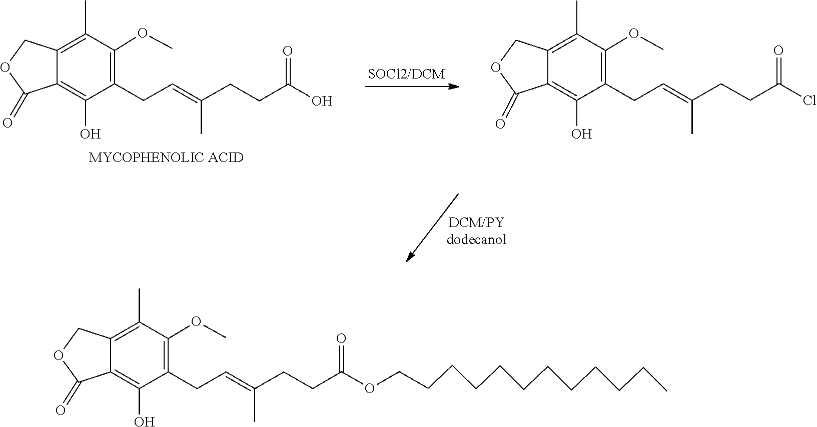 [SMILES: COc1c(C)c2c(c(O)c1C/C=C(\C)CCC(=O)O)C(=O)OC2 ; COc1c(C)c2c(c(O)c1C/C=C(\C)CCC(=O)Cl)C(=O)OC2 ; CCCCCCCCCCCCOC(=O)CC/C(C)=C/Cc1c(O)c2c(c(C)c1OC)COC2=O]

Mycophenolic acid (700 mg; 2.18 mmol) was dissolved in anhydrous dichloromethane (20 ml). Thionyl chloride (0.48 ml; 3 mol/eq) was added and the solution was stirred at room temperature for 5 hours. The solvent was removed under vacuum. The residue was dissolved in 10 ml of anhydrous dichloromethane and added dropwise to a solution of 1-dodecanol (404 mg; 1 mol/eq) in anhydrous dichloromethane (10 ml) and pyridine (1.75 ml; 10 mol/eq.). The resulting mixture was stirred at room temperature for 12 hours under nitrogen atmosphere, then checked by HPLC-MS.

Water was added, the two phases were separated, and the organic phase was further washed with 4% HCl solution, water and brine. The solvent was removed, and the residue was purified by chromatography (Isolera Biotage; Sfar column; eluent: hexane/ethyl acetate 9/1). Overall yield: 13%.

### PRO-MPA-C16 Synthesis

The method set up by the present inventors for the synthesis of the molecule hexadecyl (4E)-6-(1,3-dihydro-4-hydroxy-6-methoxy-7-methyl-3-oxo-5-isobenzofuranyl)-4-methyl-4-hexenoate (PRO-MPA-C16) is shown in the diagram below:

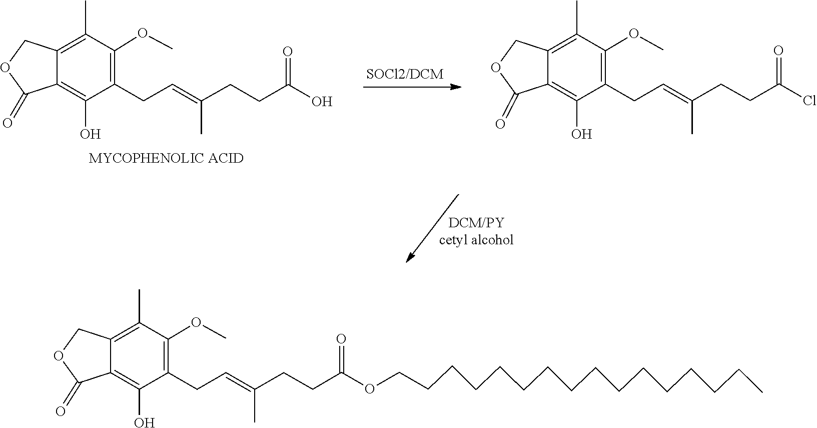 [SMILES: COc1c(C)c2c(c(O)c1C/C=C(\C)CCC(=O)O)C(=O)OC2 ; COc1c(C)c2c(c(O)c1C/C=C(\C)CCC(=O)Cl)C(=O)OC2 ; CCCCCCCCCCCCCCCCOC(=O)CC/C(C)=C/Cc1c(O)c2c(c(C)c1OC)COC2=O]

In short, mycophenolic acid (1 g; 3.12 mmol) was dissolved in anhydrous dichloromethane (40 ml). Thionyl chloride (0.68 ml; 3 mol/eq) was added and the solution was stirred at room temperature for 5 hours. The solvent was removed under vacuum. The residue was dissolved in 10 ml of anhydrous dichloromethane and added dropwise to a solution of 1-hexadecanol (830 mg; 1 mol/eq) in anhydrous dichloromethane (10 ml) and pyridine (2.15 ml; 10 mol/eq). The resulting mixture was stirred at room temperature for 12 hours under nitrogen atmosphere, then checked by HPLC-MS. Water was added, the two phases were separated, and the organic phase was further washed with 4% HCl solution, water and brine. The solvent was removed, and the residue was purified by chromatography (Isolera Biotage; Sfar column; eluent: hexane/ethyl acetate 9/1). Overall yield: 25%.

The mycophenolic acid prodrug molecules obtained by the synthesis methods described above were characterized by 1H-NMR and HPLC-MS. As regards the 1H-NMR technique, one sample aliquot was dissolved in deuterated chloroform and analysed by nuclear magnetic resonance (300 MHz). HPLC assays were performed using the Phenomenex Gemini-NX C18 3 μm 150-2.0 mm chromatographic column. The samples were dissolved in the mobile phase Acetonitrile/Water 1:1. For sample identification, the HPLC was combined with a mass spectrometer (MS ES+). Sample purity was assessed by analysing the chromatographic peak areas obtained by HPLC assay.

Table 1 below shows the physicochemical characteristics of the above mycophenolic acid prodrug molecules.

TABLE 1

| | | MOLECULAR | | |
|---|---|---|---|---|
| | | WEIGHT | Calculated | |
| | STATE | (g/mol) | LogP | PURITY (%) |
| PRO- | Colourless | 390.47 | 5.51 | 97.4 |
| MPA-C5 | oil | | | |
| PRO- | Colourless | 432.55 | 7.11 | 97.4 |
| MPA-C8 | oil | | | |
| PRO- | Colourless | 488.65 | 9.23 | 99.5 |
| MPA-C12 | oil | | | |
| PRO- | White solid | 544.78 | 11.36 | 98.8 |
| MPA-C16 | | | | |

### Example 3: Preparation of PRO-MPA-C16 Having the R¹ Portion Derivatized with Polyethylene Glycol (PEG)

### PRO-MPA-C16-OPEG Synthesis

The method set up by the present inventors for the synthesis of the molecule hexadecyl (4E)-6-(1,3-dihydro-4-carbonylpolyethylene glycol monomethoxy-6-methoxy-7-methyl-3-oxo-5-isobenzofuranyl)-4-methyl-4-hexenoate (PRO-MPA-C16-OPEG) is shown in the diagram below:

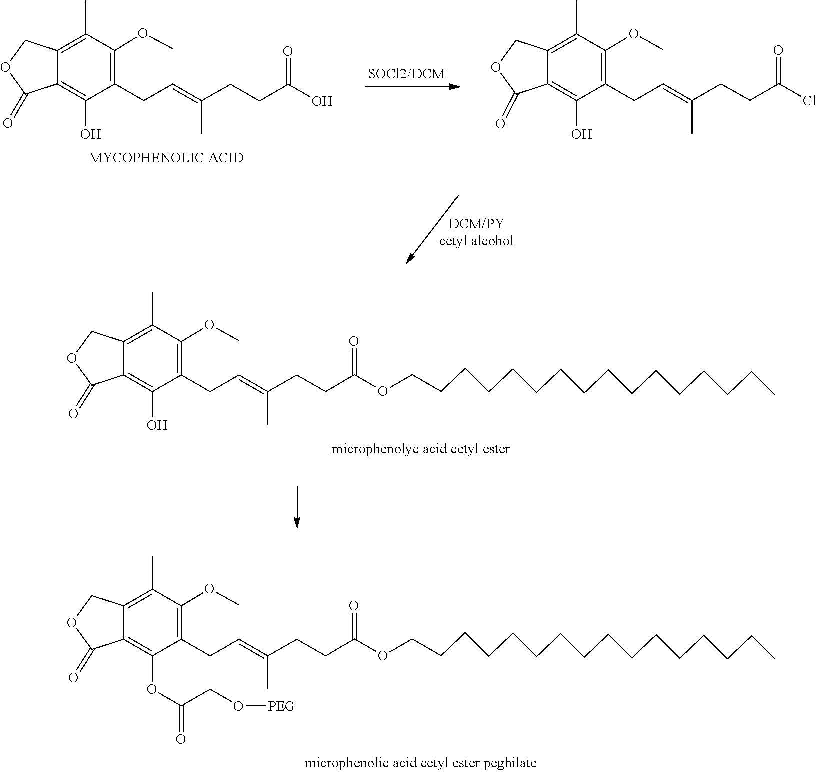

In short, mycophenolic acid (1 g; 3.12 mmol) was dissolved in anhydrous dichloromethane (40 ml). Thionyl chloride (0.68 ml; 3 mol/eq) was added and the solution was stirred at room temperature for 5 hours. The solvent was removed under vacuum. The residue was dissolved in 10 ml of anhydrous dichloromethane and added dropwise to a solution of 1-hexadecanol (830 mg; 1 mol/eq) in anhydrous dichloromethane (10 ml) and pyridine (2.15 ml; 10 mol/eq). The resulting mixture was stirred at room temperature for 12 hours under nitrogen atmosphere, then checked by HPLC-MS. Water was added, the two phases were separated, and the organic phase was further washed with 4% HCl solution, water and brine. The solvent was removed, and the residue was purified by chromatography (Isolera Biotage; Sfar column; eluent: hexane/ethyl acetate 9/1). Overall yield: 25%.

Mycophenolic acid cetyl ester (100 mg; 0,184 mmol) was dissolved in dichloromethane (5 ml) and HOBT (0.2 mol/eq; 5 mg), diisopropylethylamine (2 mol/eq; 50 mg), EDC·HCl (1.5 mol/eq; 53 mg) and PEG-COOH (MW 970 g/mol; 196 mg; 1.1 mol/eq) were added. The mixture was stirred at room temperature at 25° C. for 24 hours, washed with water and the phases were separated. The organic phase was evaporated under vacuum. The oily residue was purified using a chromatographic column (Isolera Biotage; Sfar column; eluent: dichloromethane/methanol 95/5. Yield: 35%.

The mycophenolic acid prodrug molecules obtained by the synthesis methods described above were characterized with the methods set out in Example 2.

Table 2 below shows the physicochemical characteristics of the above PRO-MPA-C16-OPEG molecule.

TABLE 2

| | | MOLECULAR | | |
|---|---|---|---|---|
| | | WEIGHT | Calculated | |
| | STATE | (g/mol) | LogP | PURITY (%) |
| PRO- | Colourless | 1496.78 | 3.57 | 97.0 |
| MPA- | oil | | | |
| C16- | | | | |
| OPEG | | | | |

### Example 4: Preparation of the Nanoparticles According to the Invention

For the preparation of the nanoparticles of the invention, the present inventors employed the solvent injection method, also referred to as nanoprecipitation. In short, mycophenolic acid prodrug molecules as described above were solubilized in a known volume of water-miscible organic solvent and the resulting mixture was injected by means of an automatic injector into a known volume of MilliQ water. In a typical preparation, the organic solvent-to-water ratios were 1:10 (v/v). Once the injection phase was completed, the organic solvent was removed by vacuum evaporation and the final volume of water was adjusted based on the desired concentration of the above molecules.

In greater detail, 2.5 mg of PRO-MPA-C16 were dissolved in 1 ml of ethanol and the resulting organic mixture was injected into 10 ml of MilliQ water (40° C.) under stirring. The resulting suspension was then subjected to vacuum evaporation to remove the ethanol and part of the water up to a final volume of 5 ml in order to obtain a final concentration of PRO-MPA-C16 nanoparticles of 0.5 mg/ml.

The sample was then centrifuged for 10 minutes at 4000 RPM to remove any non-self-assembled clusters and/or molecules and subsequently the supernatant consisting of nanoparticles according to the invention was collected and stored at 4° C.

By means of the method as described above, the present inventors prepared nanoparticles including the four different mycophenolic acid prodrug molecules, namely the PRO-MPA-C5, PRO-MPA-C8, PRO-MPA-C12 and PRO-MPA-C16 nanoparticles. Tables 3 and 4 below show some chemical-physical parameters measured for said nanoparticles, immediately after their preparation (T=0) and after one week, i.e., 168 hours (T=1 week), respectively.

TABLE 3

| | | | Zeta potential | |
|---|---|---|---|---|
| | | Size (nm +/− SD) | (mV) | PDI |
| T = 0 | PRO- | 283.1 ± 7.6 | −32.5 ± 0.4 | 0.188 |
| | MPA-C5 | | | |
| | PRO- | 258.7 ± 3.9 | −27.7 ± 0.6 | 0.045 |
| | MPA-C8 | | | |
| | PRO- | 116.4 ± 1.5 | −20.9 ± 0.1 | 0.133 |
| | MPA-C12 | | | |
| | PRO- | 130.8 ± 2.5 | −15.8 ± 4.7 | 0.121 |
| | MPA-C16 | | | |

TABLE 4

| | | | Zeta potential | |
|---|---|---|---|---|
| | | Size (nm +/− SD) | (mV) | PDI |
| T = 1 week | PRO- | 388.6 ± 9.7 | −30.2 ± 1.8 | 0.352 |
| | MPA-C5 | | | |
| | PRO- | 602.4 ± 8.6 | −35.1 ± 0.6 | 0.239 |
| | MPA-C8 | | | |
| | PRO- | 218.4 ± 0.5 | −45.1 ± 1.0 | 0.073 |
| | MPA-C12 | | | |
| | PRO- | 135.3 ± 2.9 | −20.2 ± 3.8 | 0.168 |
| | MPA-C16 | | | |

As can be seen from the size data given in Tables 3 and 4, the nanoparticles comprising PRO-MPA-C16 appear to be more stable. In fact, after a week, no significant increase in size is observed, unlike what is observed for nanoparticles obtained with the other prodrug molecules.

The present inventors also prepared nanoparticles of the invention, in particular PRO-MPA-C16 nanoparticles, using a functionalized polyethylene glycol phospholipid conjugate (DSPE-PEG2000) as a surfactant component.

In this approach, 2.5 mg of PRO-MPA-C16 molecules and 0.250 mg of DSPE-PEG2000 were dissolved in 1 ml of ethanol and the resulting organic mixture was injected into 10 ml of MilliQ water (40° C.) under stirring. The resulting suspension was then subjected to vacuum evaporation to remove the ethanol and part of the water up to a final volume of 5 ml in order to obtain a final concentration of PRO-MPA-C16 nanoparticles of 0.5 mg/ml.

The sample (PRO-MPA-C16-P) was then centrifuged for 10 minutes at 4000 RPM to remove any non-self-assembled prodrug clusters and/or molecules and subsequently the supernatant consisting of nanoparticles of the invention was collected and stored at 4° C.

The present inventors also prepared nanoparticles of the invention consisting of PRO-MPA-C16 and PRO-MPA-C16-OPEG.

In this approach, 2.5 mg of PRO-MPA-C16 molecules and 0.250 mg of PRO-MPA-C16-OPEG molecules were dissolved in 1.667 ml of ethanol and the resulting organic mixture was injected into 5 ml of the aqueous buffer [tris(hydroxymethyl)methylamino]propanesulfonic acid (TAPS) under stirring. The resulting suspension was then subjected to vacuum evaporation to remove the ethanol and part of the water up to a final volume of 3.5 ml in order to obtain a final concentration of nanoparticles of 0.79 mg/ml.

The sample (PRO-MPA-C16-PRO-MPA-C16-OPEG) was then centrifuged for 10 minutes at 4000 RPM to remove any non-self-assembled prodrug clusters and/or molecules and subsequently the supernatant consisting of nanoparticles of the invention was collected and stored at 4° C.

The results of the characterization of PRO-MPA-C16 nanoparticles also comprising the surfactant DSPE-PEG2000, and of PRO-MPA-C16 nanoparticles also comprising PRO-MPA-C16-OPEG are given in Table 5 below:

TABLE 5

| | | | Zeta | |
|---|---|---|---|---|
| | | Size | potential | |
| | | (nm +/− SD) | (mV) | PDI |
| T = 0 | PRO-MPA- | 79.6 ± 1.7 | −25.8 ± 2.4 | 0.146 |
| | C16-P | | | |
| | PRO-MPA- | 95.7 ± 3.3 | −27.2 ± 0.8 | 0.157 |
| | C16-PRO- | | | |
| | MPA-C16- | | | |
| | 0PEG | | | |

### Example 5: Encapsulation of Lipophilic Active Ingredients in the Nanoparticles of the Invention

The present inventors encapsulated, through a physical encapsulation process, lipophilic active ingredients within the nanoparticles of the invention using the solvent injection method, also referred to as nanoprecipitation, as given in Example 4. In a first example, the Galunisertib molecule was encapsulated within the PRO-MPA-C16 nanoparticles.

In this approach, 5 mg of PRO-MPA-C16 molecules and 1 mg of Galunisertib were dissolved in 1.667 ml of ethanol and the resulting organic mixture was injected into 5 ml of the aqueous buffer [tris(hydroxymethyl)methylamino]propanesulfonic acid (TAPS) under stirring. The resulting suspension was then subjected to vacuum evaporation to remove the ethanol and part of the water up to a final volume of 3.5 ml in order to obtain a final concentration of PRO-MPA-C16 nanoparticles of 1.43 mg/ml and a final Galunisertib concentration of 0.28 mg/ml.

The sample (PRO-MPA-C16-GAL) was then centrifuged for 10 minutes at 4000 RPM to remove non-self-assembled prodrug molecules and/or non-encapsulated active ingredient (Galunisertib), and subsequently the supernatant consisting of nanoparticles of the invention was collected.

In a second example, the active ingredient Nintedanib was encapsulated within the PRO-MPA-C16 nanoparticles. In this approach, 3.5 mg of PRO-MPA-C16 molecules and 0.350 mg of Nintedanib were dissolved in 1.667 ml of ethanol and the resulting organic mixture was injected 5 ml into of the aqueous buffer [tris(hydroxymethyl)methylamino]propanesulfonic acid (TAPS) at pH 9.0 under stirring. The resulting suspension was then subjected to vacuum evaporation to remove the ethanol and part of the water up to a final volume of 3.5 ml in order to obtain a final concentration of PRO-MPA-C16 nanoparticles of 1 mg/ml and a final concentration of Nintedanib of 0.1 mg/ml.

The sample (PRO-MPA-C16-NTB) was then centrifuged for 10 minutes at 4000 RPM to remove non-self-assembled prodrug molecules and/or non-encapsulated active ingredient (Nintedanib), and subsequently the supernatant consisting of nanoparticles of the invention was collected.

The results of the characterization of the nanoparticles obtained in Example 5 are given in Table 6 below:

TABLE 6

| | | | Zeta | |
|---|---|---|---|---|
| | | Size | potential | |
| | | (nm +/− SD) | (mV) | PDI |
| T = 0 | PRO-MPA- | 93.6 ± 3.1 | −18.7 ± 5.7 | 0.153 |
| | C16-GAL | | | |
| | PRO-MPA- | 143.5 ± 9.7 | −20.2 ± 3.8 | 0.170 |
| | C16-NTB | | | |

### Example 6: Assessment of the Critical Micelle Concentration (CMC)

The critical micelle concentration (CMC) is the concentration at which a number of monomers cluster, leading to the formation of micelles. The CMC relating to the mycophenolic acid prodrug molecules described above, more specifically the PRO-MPA-C16 molecules, was determined using the multi-angle dynamic light scattering technology based on the measurement of the scattering intensity. The scattering intensity is the intensity of scattered light as a result of the interaction of the light beam with the nanoparticles. This is a function of the concentration of monomers in solution. As the monomers cluster to form micelles, the scattering intensity increases significantly.

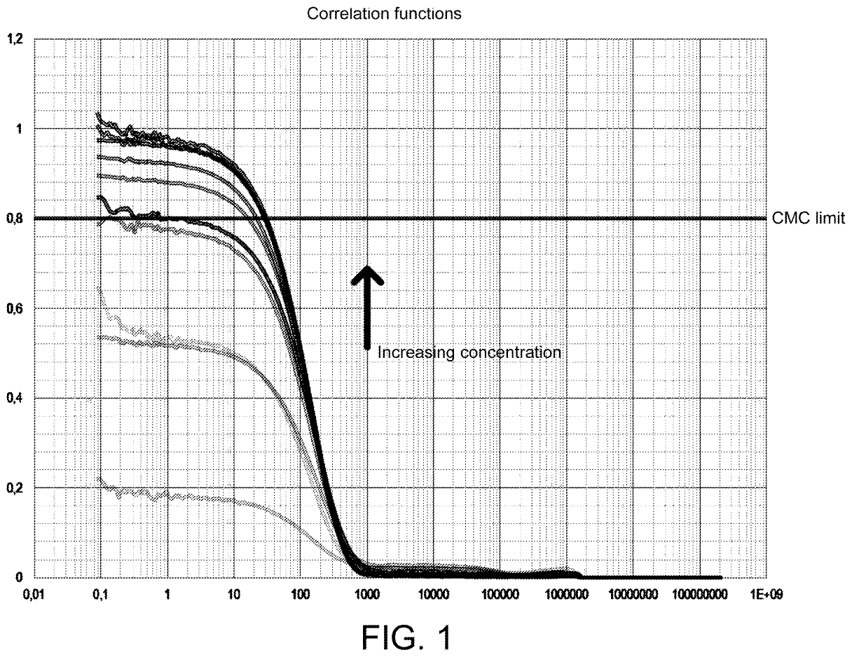

The diagram in FIG. 1, showing the correlograms obtained from the analysis of the sample of PRO-MPA-C16 nanoparticles in milliQ water at 25° C. at each tested concentration, shows the trend of the intercept of the correlation function, which increases as the tested concentration increases. The horizontal line represents the CMC limit of 0.8 g₂-1 for the formation of colloidal structures/CMC.

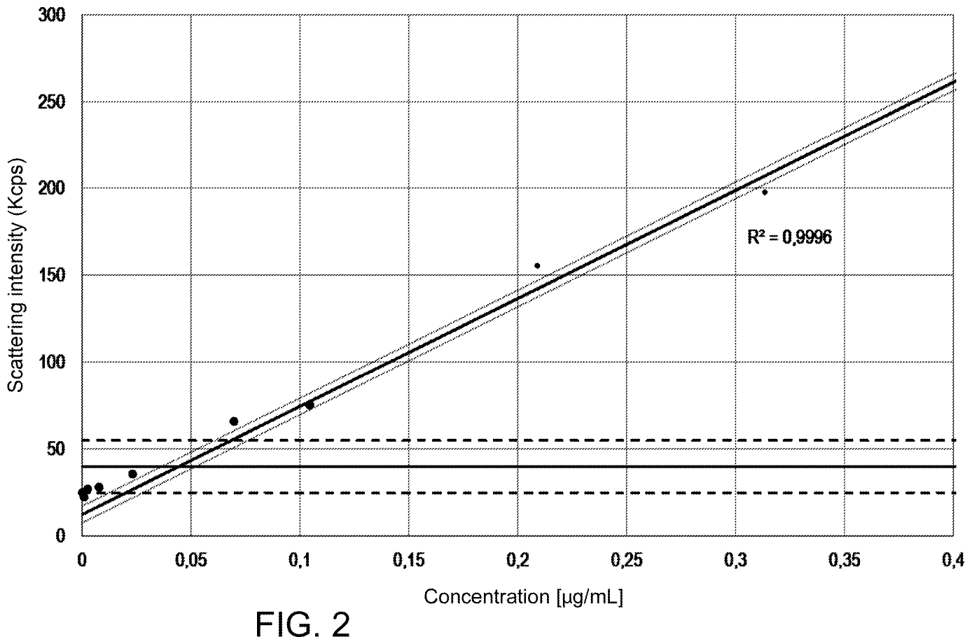

The diagram in FIG. 2 shows the scattering intensity (“derived count rate”) as a function of the concentration of PRO-MPA-C16 nanoparticles tested, with its trend line.

The CMC value appears to be 0.075±0.025 μg/ml from the combination of the test results of the correlograms and scattering intensities, as a function of the PRO-MPA-C16 concentrations tested. This calculated experimental value is considerably lower than traditional micellar systems used in the pharmaceutical industry such as, for example, monomethoxy poly(ethylene glycol)-block-poly(D,L-lactide) used in GENOXOL PM, which has a CMC of 44 μg/ml, and Tween 80 used in ESTRASORB, which has a CMC of 13-15 μg/ml.

Advantageously, such a low CMC value can ensure high stability of the nanoparticles of the invention after dilution in biological fluids.

### Example 7: Assessment of the In Vitro Enzymatic Inhibition Effects of the Nanoparticles of the Invention

As is known, mycophenolic acid is a potent inhibitor of the enzyme Inosine Monophosphate Dehydrogenase (IMPDH), which is critical for the synthesis of purine nucleotides.

In order to assess the activity of the nanoparticles according to the present invention, in particular the ability to block the aforementioned enzyme, the inventors carried out in vitro studies on human bronchial epithelial cells (16HBE140).

During the experiments, 16HBE140 cells were placed in contact with three different concentrations of nanoparticles of the invention consisting of PRO-MPA-C16 (450-150-50 nM), for periods of 6-4-2 hours. The activity of the enzyme IMPDH was measured by means of a specific test (IMPDH assay from Assay Genie). Samples of 16HBE140 cells treated with mycophenolic acid at the same concentrations and at the same times as above were used as a control.

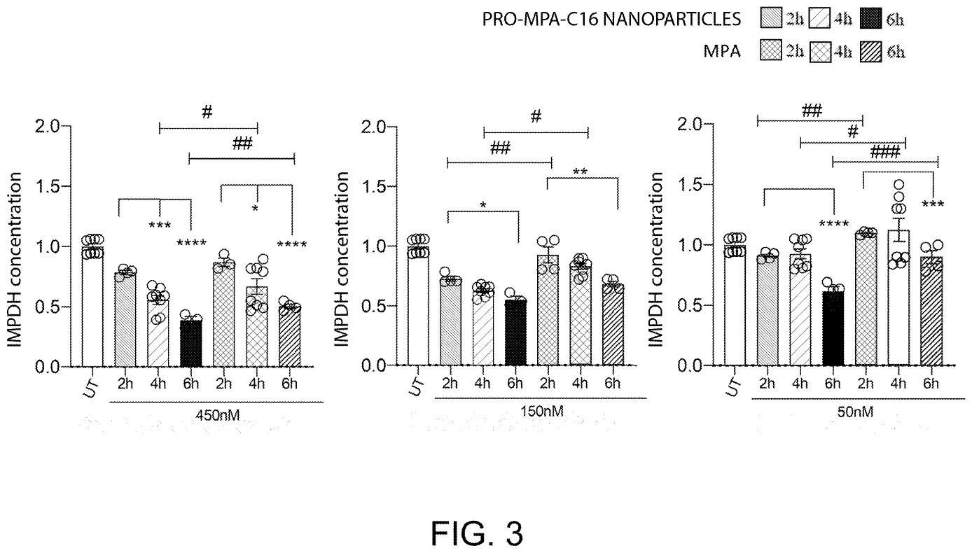

As shown in the diagrams in FIG. 3, which show the decrease in the IMPDH enzyme in cells after treatment with PRO-MPA-C16 nanoparticles or mycophenolic acid at increasing concentrations and times, the nanoparticles of the invention induced a dose-dependent decrease in IMPDH levels. The enzymatic activity of IMPDH was inversely proportional to the concentration of MPA.

In the diagrams in FIG. 3, the data are expressed as the mean±SEM and the amount of IMPDH enzyme was normalized to untreated cells. The statistical significance values *P<0.05, **P<0.01, ***P<0.001, ****P<0.0001 vs 2 h, #P<0.05, ##P<0.01, ###P<0.001 PRO-MPA-C16 vs MPA were calculated with the one-way ANOVA test, followed by Bonferroni's posthoc test.

Importantly, the inhibition of the IMPDH enzyme was significantly higher when the cells were treated with PRO-MPA-C16 nanoparticles than with MPA. The maximum enzyme inhibition effect occurred after 6 hours of treatment.

### Example 8: Assessment of the In Vitro Enzymatic Inhibition Effects of the Nanoparticles of the Invention in the Presence of Mucus

In order to verify whether the nanoparticles of the invention are also able to perform their activity in physiological secretions, especially in particularly dense and viscous mucous secretions, the inventors reproduced the experiments as described in the previous paragraph on human bronchial epithelial cells in the presence of a layer of sputum from patients suffering from bronchiectasis/cystic fibrosis.

The results of these studies, shown in the diagram in FIG. 4, show a significantly higher inhibition effect of PRO-MPA-C16 nanoparticles on the IMPDH enzyme compared to the mycophenolic acid control already after two hours of treatment.

This indicates that the nanoparticles of the invention are surprisingly capable of penetrating/permeating viscous mucous secretions, while maintaining their enzymatic inhibition activity mediated by their ability to release mycophenolic acid in a localized manner.

### Example 9: Assessment of the In Vitro Cell Proliferation and Enzymatic Activity Inhibition Effects of the Nanoparticles of the Invention on Immune Cells

The inventors also carried out in vitro studies on immune cells (PBMCs and T-Jurkat) with the aim of assessing the ability of the nanoparticles according to the present invention to inhibit the proliferation of these cells as well as the enzymatic activity of the target enzyme IMPDH.

The cell proliferation inhibition test was performed on the PBMC cell line. In order to assess the ability of the inventive PRO-MPA-C16 nanoparticles to inhibit cell proliferation, PMBC cells were pre-stimulated with 1 μM ionomycin and 2.5 μg/ml phytohaemagglutinin (PHA) in order to induce the proliferation thereof.

The cells were then treated for 72 hours with the nanoparticles of the invention consisting of PRO-MPA-C16, used at two different concentrations (2 μM and 10 μM). Mycophenolic acid (MPA) at a concentration of 10 μM was used as a comparison. Cell proliferation was assessed using a method (CellTiter-Glo®, Promega) that determines the number of viable cells by quantifying adenosine triphosphate (ATP) levels.

As shown in the diagram in FIG. 5, which shows decreased cell proliferation after treatment with PRO-MPA-C16 nanoparticles or mycophenolic acid at 72 hours, the nanoparticles of the invention inhibited cell proliferation in a dose-dependent manner.

A second study assessed the ability of PRO-MPA-C16 nanoparticles to inhibit the enzyme IMPDH in order to demonstrate that the above cell proliferation inhibition was actually mediated by the inhibition of this enzyme. Tests were performed on PMBC (stimulated and unstimulated) and T-Jurkat (unstimulated) cell lines.

The cells were treated for 24 hours with PRO-MPA-C16 nanoparticles at two different concentrations (2 μM and 10 μM). MPA at a concentration of 10 μM was used as a comparison. The enzyme IMPDH was subsequently extracted and assessed for its ability to convert a substrate into a given product. This test was carried out using a special kit (IMPDH Assay Kit, Biomedical Research Service, University at Buffalo, USA) which assesses the ability of the enzyme IMPDH to convert the iodine-nitro-tetrazolium (INT) substrate into the formazan product. The enzymatic activity is proportional to the amount of formazan produced. The latter is assessed by spectrophotometric measurement at a wavelength of 492 nm.

As shown in the diagrams in FIG. 6, which show the decreased activity of the IMPDH enzyme in PBMC and T-Jurkat cells after treatment for 24 hours with PRO-MPA-C16 nanoparticles or mycophenolic acid, the nanoparticles of the invention induced a dose-dependent decrease of IMPDH activity. The enzymatic activity of IMPDH was inversely proportional to the concentration of the PRO-MPA-C16 nanoparticles.

In the diagrams in FIGS. 5 and 6, the data are expressed as the mean±SEM. The data shown in FIG. 5 are normalized to the biological control (unstimulated cells). The statistical significance values *P<0.05, **P<0.01 vs untreated stimulated cells were calculated with the one-way ANOVA test.

In FIG. 6, the statistical significance values * p<0.05 vs untreated cells; § p<0.05 vs 10 μM MPA were calculated with the Kruskal-Wallis test followed by Dunnett's post hoc test.

### Example 10: Assessment of the In Vivo Effectiveness of the Nanoparticles of the Invention in an SSc-ILD Mouse Model

A mouse model of systemic sclerosis-associated interstitial lung disease (SSc-ILD) was used to assess the in vivo effectiveness of the nanoparticles of the invention. This model, widely described in the literature (Ravanetti et al. “SSC-ILD mouse model induced by osmotic minipump delivered bleomycin: effect of Nintedanib”, Scientific Reports. 2021; 11.18513), induces subacute-chronic fibrosis in the lungs and skin through the release of bleomycin (BLM) by osmotic mini-pumps. In particular, osmotic mini-pumps [ALZET 1007D; DURECT, (release rate 0.5 μl/h for 7 days), Cupertino, California, USA] containing 100 μl of saline or BLM (60 U/kg dissolved in saline) are implanted in female C57BL/6 mice of 2-3 months of age, in a subcutaneous pocket obtained in the intrascapular region and removed after 8 days. On day 14 the treatment begins and continues until the animals are sacrificed on day 21.

Treatment of the animals was performed by intratracheally administering twice a day, via the Penn-Century MicroSprayer® Aerosolizer-Model IA-1C and FMJ-250 High Pressure Syringe, 50 μl of vehicle (TAPS buffer) or 50 μl of the inventive nanoparticles consisting of PRO-MPA-C16. The latter were administered at a concentration of 2.7 mg/kg body weight twice a day (the concentration refers to the actual amount of MPA present in the nanoparticles).

Specifically, 4 groups of animals were used:
1) Naïve (healthy) group treated with the vehicle (Naïve+TAPS);
2) Naïve (healthy) group treated with PRO-MPA-C16 nanoparticles (Naïve+PRO-MPA-C16 nanoparticles);
3) BLM (diseased) group treated with the vehicle (BLM+TAPS);
4) BLM (diseased) group treated with PRO-MPA-C16 nanoparticles (BLM+PRO-MPA-C16 nanoparticles).

The aeration state of the lungs was assessed at days 0, 14 and 21 through the micro-computed tomography (micro-CT) technique using the PerkinElmer Quantum FX μCT instrument at 90 kV, 88 uA, 36 FOV for 4 minutes, combined with the Analyze 14.0 software. Image sequences were generated and subsequently filtered and converted from grey levels to CT numbers (Hounsfield Units—HU). The Hounsfield Units scale is a unit of measurement scale used to quantitatively describe the radiodensity.

The conversion is a linear transformation that involves setting-1000 HU as the density of air and 0 HU as the density of water. A semi-automatic segmentation is used to highlight airways and lungs. For quantitative assessment of the lung parenchyma, HU clinical ranges were applied to HU images of the lung to define it as normally aerated [(−900, −500) HU] or poorly aerated [(−500, −100) HU] (Gattinoni et al. in “What has computed tomography taught us about the acute respiratory distress syndrome?”, Am J Respir Crit Care Med. 2001; 164: 1701-1711. Ravanetti et al. “SSC-ILD mouse model induced by osmotic minipump delivered bleomycin: effect of Nintedanib”, Scientific Reports. 2021; 11.18513).

The two compartments with different degrees of aeration are expressed as a percentage of the total lung volume. Poorly aerated tissue will refer to a low gas-to-tissue ratio and has been used to quantify the progression of pulmonary fibrosis and assess the effectiveness of the PRO-MPA-C16 nanoparticles of the invention.

FIG. 7 (A) shows the lung aeration levels of all the animals used in the experiment on Day 0 (data are given as the mean±SEM). Specifically, both healthy (naïve) animals and those that will subsequently receive Bleomycin via osmotic minipump (BLM) show comparable levels of lung aeration. FIG. 7 (B) shows the lung aeration levels on Day 14 (data are given as the mean±SEM; **P<0.01 vs. Naïve (unpaired t-test). It can be seen that healthy (naïve) mice show aeration levels completely comparable to those seen on Day 0, whereas diseased mice (BLM) show a decrease in the levels of well-aerated tissue and an increase in the levels of poorly aerated tissue, which are typical clinical manifestations of the disease. This clearly indicates that treatment with BLM induced the disease. FIG. 7 (C) shows the lung aeration levels of the 4 groups of animals on Day 21 (data are given as the mean±SEM;*P<0.05 vs. BLM (ANOVA with Dunnet Post-Test); **P<0.01 vs. BLM (ANOVA with Dunnet Post-Test). It can be seen that the diseased group that has been treated with PRO-MPA-C16 nanoparticles (BLM+PRO-MPA-C16 nanoparticles) shows aeration levels completely comparable to those of the two naïve groups (NAIVE+TAPS and NAIVE+PRO-MPA-C16 nanoparticles). In contrast, the untreated diseased group (BLM+TAPS) shows lung aeration levels completely comparable to those observed on Day 14.

The data indicated in FIG. 7 (C) clearly show that treatment of diseased mice with PRO-MPA-C16 nanoparticles is particularly effective as it restores the aeration state of the lungs to levels completely comparable to those of non-diseased (naïve) mice. In particular, it can be observed that, following treatment, there is an increase in the percentage of well-aerated lung tissue and a consequent decrease in poorly aerated tissue.

Importantly, this surprising remission of the disease is achieved by administering PRO-MPA-C16 nanoparticles via the intratracheal route, at a dosage that is approximately 28 times smaller than the maximum dosage used via the oral route reported in the literature (Wollin et al. “The effect of nintedanib versus mycophenolate mofetil in the Fra2 mouse model of systemic sclerosis-associated interstitial lung disease”, Clin Ex Rheumatol. 2021; 39. S134-S141).

It is also interesting to note that the treatment described above was carried out by administering the inventive nanoparticles consisting of PRO-MPA-C16 which exhibit the peculiarity of being formulated without the use of excipients, thereby maximising the drug loading (weight ratio between active ingredient and excipients). This is a major advantage in inhalation administration, as it allows high doses of the active ingredient to be administered. In addition, local administration at the pulmonary level allows for a significant reduction in dosages, resulting in a reduction in the total immunosuppressive load and the side effects associated therewith.

1. A nanoparticle consisting of a plurality of therapeutic molecules arranged in the form of a spherical micelle, the therapeutic molecules having the general formula (I) [symbol] 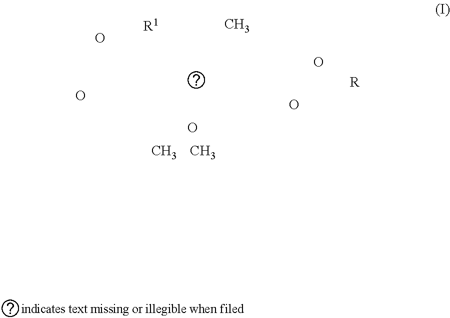
wherein
R is a linear or branched, saturated or unsaturated, alkyl chain containing from 5 to 22 carbon atoms, and
R¹ is —OH, optionally conjugated with a conjugation molecule selected from the group consisting of polyethylene glycol (PEG), triethylene glycol (TEG), hyaluronic acid, sugars, proteins, peptides, antibodies, messenger RNAs (mRNAs), small interfering RNAs (siRNAs), microRNAs (miRNAs), aptamers, and any combination thereof.

2. The nanoparticle of claim 1, wherein the nanoparticle has a diameter ranging from 30 nm to 700 nm, and/or the zeta potential of the nanoparticle ranges from −5 mV to −60 mV.

3. (canceled)

4. The nanoparticle of claim 1, wherein the nanoparticle contains internally a lipophilic therapeutic agent.

5. A pharmaceutical composition comprising the nanoparticle of claim 1, and at least one pharmaceutically acceptable vehicle, excipient and/or diluent.

6. The pharmaceutical composition of claim 5, which is in a pharmaceutical form suitable for administration via topical, oral, rectal, sublingual, intravenous, intraarterial, transcutaneous, intramuscular, intradermic, subcutaneous, intraperitoneal, ocular, inhalation, and intratracheal route.

7. A method for treating a subject affected by an autoimmune disease and/or a fibrotic lung disease, the method comprising administering to the subject an effective amount of the nanoparticle of claim 1 or of a pharmaceutical composition comprising the nanoparticle of claim 1, and at least one pharmaceutically acceptable vehicle, excipient and/or diluent.

8. (canceled)

9. A method for treatment and/or prevention of organ rejection in a subject in need thereof, the method comprising administering to the subject an effective amount of the nanoparticle of claim 1 or of a pharmaceutical composition comprising the nanoparticles of claim 1, and at least one pharmaceutically acceptable vehicle, excipient and/or diluent.

10. A method for the production of a nanoparticle consisting of a plurality of therapeutic molecules arranged in the form of a spherical micelle, the therapeutic molecules having the general formula (I) 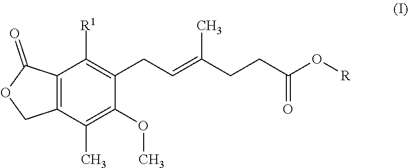
wherein
R is a linear or branched, saturated or unsaturated, alkyl chain containing from 5 to 22 carbon atoms, and
R¹ is —OH, optionally conjugated with a conjugation molecule selected from the group consisting of polyethylene glycol (PEG), triethylene glycol (TEG), hyaluronic acid, sugars, proteins, peptides, antibodies, messenger RNAs (mRNAs), small interfering RNAs (siRNAs), microRNAs (miRNAs), aptamers, and any combination thereof, the method comprising:
(i) mixing the plurality of therapeutic molecules in a polar solvent, thereby obtaining a first mixture, wherein the polar solvent is not water;
(ii) adding the first mixture to a predetermined amount of water under stirring, so as to obtain a second mixture, wherein the predetermined amount of water is at least 65% by volume of a total volume of the second mixture; and
(iii) removing the polar solvent from the second mixture, thereby obtaining the nanoparticle consisting of a plurality of therapeutic molecules arranged in the form of a spherical micelle, in an aqueous suspension.

11. A method of reducing lymphocyte proliferation in the lungs of a subject in need thereof, the method comprising administering to the subject an effective amount of the nanoparticle of claim 1 or of a pharmaceutical composition comprising the nanoparticle of claim 1, and at least one pharmaceutically acceptable vehicle, excipient and/or diluent, wherein the subject is affected by an autoimmune disease and/or a fibrotic lung disease.

12. A method of reducing lymphocyte proliferation in the lungs of a subject in need thereof, the method comprising administering to subject an effective amount of the nanoparticle of claim 1 or of a pharmaceutical composition comprising the nanoparticle of claim 1, and at least one pharmaceutically acceptable vehicle, excipient and/or diluent, wherein the subject has undergone organ transplantation.

13. A method of improving lung aeration in a subject in need thereof, the method comprising administering to the subject an effective amount of the nanoparticle of claim 1 or of a pharmaceutical composition comprising the nanoparticle of claim 1, and at least one pharmaceutically acceptable vehicle, excipient and/or diluent.

14. The method of claim 7, wherein the autoimmune disease and/or the fibrotic lung disease is selected from the group consisting of systemic sclerosis-associated interstitial lung disease (SSC-ILD), lymphangioleiomyomatosis (LAM), sarcoidosis, and bronchiolitis obliterans syndrome (BOS).

15. The method of claim 9, wherein the subject has undergone lung transplantation.

16. The method of claim 13, wherein the subject is affected by an autoimmune disease and/or a fibrotic lung disease.

17. The nanoparticle of claim 1, wherein the sugars are mannose and trehalose.

18. The nanoparticle of claim 2, wherein the nanoparticle has a diameter ranging from 100 nm to 200 nm.

19. The nanoparticle of claim 4, wherein the lipophilic therapeutic agent is selected from the group consisting of immunosuppressive agents, anti-inflammatory agents, antifibrotic agents, and antitumor agents.

20. The method of claim 11, wherein the autoimmune disease and/or the fibrotic lung disease is selected from the group consisting of systemic sclerosis-associated interstitial lung disease (SSc-ILD), lymphangioleiomyomatosis (LAM), sarcoidosis, and bronchiolitis obliterans syndrome (BOS).

21. The method of claim 12, wherein the subject has undergone lung transplantation.

22. The method of claim 16, wherein the autoimmune disease and/or the fibrotic lung disease is selected from the group consisting of systemic sclerosis-associated interstitial lung disease (SSC-ILD), lymphangioleiomyomatosis (LAM), sarcoidosis, and bronchiolitis obliterans syndrome (BOS).
